(19) United States
(12) Patent Application Publication
(10) Pub. No.: US 2024/0325474 A1
(43) Pub. Date: Oct. 3, 2024

(54) BOTANICAL EXTRACTS TO INHIBIT VIRUS INFECTION

(71) Applicants: Daniel Benjamin, Millstone Twp, NJ (US); Dianne Denommee, Cranford, NJ (US); David Lorchak, Tamaqua, PA (US)

(72) Inventors: Daniel Benjamin, Millstone Twp, NJ (US); Dianne Denommee, Cranford, NJ (US); David Lorchak, Tamaqua, PA (US)

(21) Appl. No.: 18/193,054

(22) Filed: Mar. 30, 2023

(51) Int. Cl. A61K 36/03; A61K 31/365; A61K 31/737; A61K 36/19; A61P 31/14

(52) U.S. Cl. CPC A61K 36/03; A61K 31/365; A61K 31/737; A61K 36/19; A61P 31/14; A61K 2236/13; A61K 2236/15; A61K 2236/17; A61K 2236/35

(57) ABSTRACT

The present invention provides alternate means in form of a medicinal composition comprising botanical extracts comprised of an organic liquid extract derived from Laminaria japonica and an organic liquid extract derived from Andrographis paniculate along with other components as disclosed herein for the prevention of viral binding, uptake, replication, infection, and disease caused by viral pathogens. Combined the aforementioned organic botanical extracts produce strong additivity and synergistic effect against the viral pathogens contributing towards significant reduction in viral binding, uptake, replication, infection, and disease. Also disclosed are the methods to formulate the aforementioned medicinal composition and dosage forms for use in a method for prophylactic treatment and prevention of viral binding, uptake, replication, infection, and disease caused by viral pathogens in an individual, which is also disclosed herein.

### FIELD OF THE INVENTION

The present invention generally relates to organic extracts derived from plants and plant materials referred to as botanical extracts, antiviral compositions, and viral disease. More specifically, the present invention relates to a combination of botanical extracts that can inhibit viral infection and consequent viral disease and medicinal composition comprising said combination for the prevention of viral binding, uptake, replication, infection, and disease caused by viral pathogens.

### BACKGROUND OF THE INVENTION

Viral infections remain a major worldwide cause of morbidity and mortality, and yet, many viruses are not yet curable. Among the most aggressive viral infections include Ebola virus, human immunodeficiency virus (HIV) causing AIDS (acquired immunodeficiency syndrome), influenza, SARS (severe acute respiratory syndrome), middle east respiratory syndrome (MERS), hepatitis virus, human papilloma virus, measles virus, dengue virus, and the newly emerged, COVID-19 virus causing severe acute respiratory syndrome coronavirus 2 (SARS-CoV-2), which is the reason for massive global deaths and the recent pandemic as declared so by the WHO in March 2020.

Coronaviruses nomenclature results from the crown-like, transmembrane spike protein surface (S) glycoprotein structures which represent the principal receptor binding sites of the virus. The receptor binding domain (RBD) of the homotrimer is identified as the pharmacophore responsible for initial binding of the virus surface and the angiotensin converting enzyme 2 (ACE2) receptor located in the nasopharyngeal tract in addition to other anatomical locations. Two main functional subunits of the S-glycoprotein have been identified and characterized, S1 subunit the RBD which binds the ACE2 receptor and the S2 subunit responsible for the viral-cell membrane fusion and entry. Initially the RBD binds to cell surface heparin sulfate (CSHS) which opens the RBD shifting the structure to kinetically favorable conformation allowing the binding of the favorable conformation to the ACE2 receptor. Human and animal cells are comprised of various glycans and glycoconjugates referred to as glycocalyx which is exploited as docking site for various viral pathogens including influenza, Herpes simplex virus, human immunodeficiency virus (HIV) and as previously described, coronavirus.

Coronavirus is a zoonotic virus that infects a range of mammalian species including dromedary, bats, cattle, cats and pangolin. These carriers act as an intermediate host for human transmission. Four main sub-groupings of coronaviruses have been elucidated: alpha, beta, gamma, and delta. Human coronaviruses were first identified in the mid-1960s. The seven coronaviruses that can infect people include the common human coronaviruses: 229E (alpha coronavirus) NL63 (alpha coronavirus) OC43 (beta coronavirus) HKU1 (beta coronavirus). These are responsible for mild-moderate cases of upper respiratory tract infections (URI) and rarely result in significant morbidity and mortality. Strains with increased morbidity and mortality include: MERS-COV (the beta coronavirus that causes Middle East Respiratory Syndrome, or MERS), SARS-COV (the beta coronavirus that causes severe acute respiratory syndrome, or SARS), and SARS-COV-2, responsible for the COVID-19 pandemic.

Currently, there is no approved remedy for many types or viruses, and vaccination is limited. Addition, these agents are often costly and ineffective due to viral resistance and cause side effects. Thus, despite global efforts in developing antiviral therapies and antiviral vaccines, toxicities and ineffective responses to resistant viral strains of synthetic antiviral drugs have reinforced the search of effective and alternative treatment options and that remains a need in the field of antiviral therapy. Based on the prevalence of diseases and infections caused by viral pathogens, there is a continuing need for new and alternate anti-viral compositions. With that in mind, naturally based pharmacotherapy may be a proper alternative for treating viral diseases such as based on antiviral medicinal plants and the isolated bioactive compounds. The present invention meets such a need and provides a solution thereto by assessing and developing plant-derived botanical extracts and combinations thereof for antiviral therapy.

### SUMMARY OF THE INVENTION

The present disclosure provides a combination of extracts from Laminaria japonica and Andrographis paniculata referred to as botanical extracts to prevent the binding and uptake of viral pathogens by oral and nasal epithelial cells from humans, companion animals, livestock, and wild animals. Further, the present invention discloses that the said combination produces additive, potentiating, and synergistic effects of various active ingredients in said extracts when two or more of such extracts are combined to increase the efficacy of the combination as compared with the efficacy of such a single ingredient when used alone. The present invention also relates to a method of formulating or producing the aforesaid combination, which is then useful as a prophylactic lozenge, lollipop, oral spray, topical ointment, and nasal spray, etc. It is further provided that the use of the defined combination of extracts in oral lozenges, lollipops, oral sprays, chewing gum, pressed powder dissolving tablet, lip balms, eye drops, and nasal sprays results in prevention of viral infection in human and animal subjects. Moreover, the present disclosure provides fabrics, filters, and coatings containing said combination of extracts is useful for clothing and face coverings to prevent the inhalation and contact with airborne and surface pathogens. Similarly, provided are room diffuser liquids for dispersion of antiviral organic substances into the air, against surface and airborne viral pathogens.

In an aspect of the present invention, it discloses a medicinal composition for the prevention of viral binding, uptake, replication, infection, and disease caused by viral pathogens, comprising: an organic liquid extract derived from Laminaria japonica; an organic liquid extract derived from Andrographis paniculate; at least one of purified distilled water, ethanol, and glycerol; optionally at least one buffering agent; optionally at least one excipient; optionally at least one food additive; optionally at least one stabilizer; optionally at least one surfactant; optionally at least one emollient; optionally at least one moisturizing agent; optionally at least one emulsifier; optionally at least one surfactant; optionally at least one antifoaming agent; optionally at least one solvent; optionally at least one coloring agent; optionally at least one flavoring agent; optionally at least one antimicrobial; optionally at least one preserving agent; optionally at least one vitamin; optionally at least one antioxidant; optionally at least one anti-inflammatory agent; optionally at least one keratolytic agent; optionally at least one chelating agent; optionally at least one anti-aging agent; optionally at least one antiseptic agent; optionally at least one plant-derived oil; optionally at least one gum base; optionally at least one humectant; optionally at least one sweetener; optionally at least one release agent; and optionally at least one thickening agent, wherein the medicinal composition is formulated into a dosage form for the prevention of viral binding, uptake, replication, infection, and disease. In another aspect of the present invention, for the medicinal composition as disclosed herein, the viral pathogens comprise coronaviruses, rhabdoviruses, influenza viruses, dengue viruses, severe acute respiratory syndrome coronaviruses (SARS-COV), severe acute respiratory syndrome coronavirus 1 (SARS-CoV-1), severe acute respiratory syndrome coronavirus 2 (SARS-COV-2), Middle East respiratory syndrome coronaviruses (MERS-COV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses (HSV), feline infectious peritonitis virus (FIPV), feline enteric coronavirus (FECV), rinderpest virus, foot-and-mouth disease virus (FMDV), cypoviruses (CPV), respiratory syncytial virus (RSV), human noroviruses (HuNoVs), murine norovirus (MNoV), feline calicivirus (FCV), and reoviruses.

In an aspect of the present invention, it discloses a method for preparing the medicinal composition for the prevention of viral binding, uptake, replication, infection, and disease caused by viral pathogens of claim 1, the method comprising the steps of: (i) preparing an organic liquid extract derived from Laminaria japonica and an organic liquid extract derived from Andrographis paniculate from the respective plant comprising the following steps: obtaining a fresh plant material from a growing plant, wherein the plant is selected from Laminaria japonica and Andrographis paniculate; washing the fresh plant material with purified distilled water, air-drying it to obtain an air-dried plant material, crushing the air-dried plant material into particles of 20-mesh size to obtain crushed particles; extracting an extract from crushed particles of step (b) to obtain an organic liquid extract derived from Laminaria japonica or an organic liquid extract derived from Andrographis paniculate; (ii) measuring and combining the organic liquid extract derived from Laminaria japonica and the organic liquid extract derived from Andrographis paniculate as obtained in step (i) together and with one or more of the following ingredients to obtain a first mixture: at least one of purified distilled water, ethanol, and glycerol; optionally at least one buffering agent; optionally at least one excipient; optionally at least one food additive; optionally at least one stabilizer; optionally at least one surfactant; optionally at least one emollient; optionally at least one moisturizing agent; optionally at least one emulsifier; optionally at least one surfactant; optionally at least one antifoaming agent; optionally at least one solvent; optionally at least one coloring agent; optionally at least one flavoring agent; optionally at least one antimicrobial; optionally at least one preserving agent; optionally at least one vitamin; optionally at least one antioxidant; optionally at least one anti-inflammatory agent; optionally at least one keratolytic agent; optionally at least one chelating agent; optionally at least one anti-aging agent; optionally at least one antiseptic agent; optionally at least one plant-derived oil; optionally at least one gum base; optionally at least one humectant; optionally at least one sweetener; optionally at least one release agent; and optionally at least one thickening agent; (iii) heating the first mixture obtained in step (ii) at temperature in the range of between 140° C. and 40° C. and mixing it while heating to obtain a second mixture; (iv) cooling the second mixture obtained in step (iii) to a temperature below room temperature to obtain a third mixture; (v) processing the third mixture obtained in step (iii) into a dosage form of the medicinal composition for the prevention of viral binding, uptake, replication, infection, and disease caused by viral pathogens as disclosed herein, wherein the dosage form is selected from a group consisting of oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings.

In an aspect of the present invention, it discloses a method for prophylactic treatment and prevention of viral binding, uptake, replication, infection, and disease caused by viral pathogens in an individual, the method comprising the steps of: (a) recruiting an individual; (b) testing the health and well-being of the individual to assess whether said individual is free of or has been previously exposed to or carrying a viral pathogen and/or resulting viral infection and viral disease; (c) categorizing the individual into pre-exposed or non-exposed group based on the result of testing in step (b); (d) administering or exposing the individual from step (c) to a medicinal composition as disclosed herein in a dosage form, said medicinal composition comprising an organic liquid extract derived from Laminaria japonica and an organic liquid extract derived from Andrographis paniculate; (e) testing the health and well-being of the individual as well as performing in vitro exposure studies in epithelial cells collected from said individual to test the effectivity of administering or exposing the individual to the medicinal composition in step (d), wherein the dosage form is selected from a group consisting of oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings, wherein the dosage form including fabric for clothing and face coverings, filters for clothing and face coverings, and coatings for clothing and face coverings is directed to prevent the inhalation and contact with airborne and surface viral pathogens, wherein the dosage form including diffusers is directed against surface and airborne viral pathogens, wherein the medicinal composition in the dosage form prevents the binding and uptake of viral pathogens by oral and nasal epithelial cells, and wherein additive, potentiating, and synergistic effects of various ingredients in the medicinal composition as combined increase the efficacy of a single ingredient as compared with the efficacy of the single ingredient alone, wherein the single ingredient is selected from a group consisting of the organic liquid extract derived from Laminaria japonica and the organic liquid extract derived from Andrographis paniculate.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention, and are incorporated in and constitute a part of the present invention and, together with the description, serve to explain the principle of the invention.

In the drawings,

FIG. 1 provides a graphical representation of the data obtained with an assessment experiment for in vitro potency of a main component of organic extract obtained from Laminaria japonica, i.e., Fucoidan in primary oral epithelial cells collected from healthy human volunteers as per the present disclosure.

FIG. 2 provides a graphical representation of the data obtained with an assessment experiment for in vivo dose response with a main component of organic extract obtained from Laminaria japonica, i.e., Fucoidan in primary oral epithelial cells collected from healthy human volunteers after three 200 μl sprays of saline or saline containing 0.125-0.5% Fucoidan as per the present disclosure.

FIG. 3 provides a graphical representation of the data obtained with an assessment experiment for a transmission study where healthy human volunteers (negative for COVID-19) did an oral rinse with COVID-19 causing viral pathogen, SARS-COV-2's spike protein in the form of 10 ml of a 1 μg/ml solution in saline and then coughed towards a petri dish containing primary oral epithelial cells collected from healthy human volunteers from a distance of 1 meter which is represented in this figure as the “Pre” bar graph and the same experiment was repeated after giving the volunteers a main component of organic extract obtained from Laminaria japonica, i.e., Fucoidan safe sweet lozenge which is represented in this figure as the “Post” bar graph as per the present disclosure.

FIG. 4 provides a graphical representation of the data obtained with an assessment experiment for an in vitro dose response with a main component of organic extract obtained from Andrographis paniculate, referred to as andrographis extract, where primary oral epithelial cells collected from healthy human volunteers were treated with 1 μg/ml spike protein and 0-2 μg andrographis extract for 1 hour at 37° C. as per the present disclosure.

FIG. 5 provides a schematic illustration of the mechanism of action underlying the additive, potentiating, and synergistic response produced with administration or exposure to a combination of botanical extracts consisting of the organic liquid extract derived from Laminaria japonica, referred to as the main component therein, Fucoidan, and the organic liquid extract derived from Andrographis paniculate, referred to as andrographis that is also seen with the Table 1 data showing strong additivity in the inhibition of SARS-Cov-2 spike protein uptake and consequent synergy in inhibition of SARS-COV-2 infection as per the present disclosure.

### DETAILED DESCRIPTION OF THE INVENTION

The present invention may be understood more readily by reference to the following detailed description of the invention taken in connection with the accompanying drawing figures, which forms a part of this disclosure. It is to be understood that this invention is not limited to the specific devices, medicines, systems, conditions or parameters described and/or shown herein and that the terminology used herein is for the example only, and is not intended to be limiting of the claimed invention. Also, as used in the specification including the appended claims, the singular forms ‘a’, ‘an’, and ‘the’ include the plural, and references to a particular numerical value includes at least that particular value unless the content clearly directs otherwise. Ranges may be expressed herein as from ‘about’ or ‘approximately’ another particular value. When such a range is expressed, it is another embodiment. Also, it will be understood that unless otherwise indicated, dimensions and material characteristics stated herein are by way of example rather than limitation, and are for better understanding of sample embodiment of suitable utility, and variations outside of the stated values may also be within the scope of the invention depending upon the particular application.

The terms “comprising,” “having,” “including,” and “containing” are to be construed as open-ended terms (i.e., meaning “including, but not limited to,” and permit the presence of one or more features or components) unless otherwise noted. It should be understood that while various embodiments in the specification are presented using “comprising” language, under various circumstances, a related embodiment may also be described using “consisting of” or “consisting essentially of language.

As such, the terms “a” (or “an”), “one or more,” and “at least one” can be used interchangeably herein. Furthermore, “and/or” where used herein is to be taken as specific disclosure of each of the two specified features or components with or without the other. Further, unless specifically stated or obvious from context, as used herein, the term “or” is understood to be inclusive. Also, unless specifically stated or obvious from context, as used herein, the terms “a”, “an”, and “the” are understood to be singular or plural.

It should also be understood that when describing a range of values, the characteristic being described could be an individual value found within the range. For example, “a pH from about pH 4 to about pH 6,” could be, but is not limited to, pH 4, 4.2, 4.6, 5.1, 5.5, etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH. In some embodiments, when the term “about” is used, it means the recited number plus or minus 10% of that recited number.

As used herein, the term “medicinal composition” includes any composition or formulation intended for food-grade, pharmaceutical, over-the-counter, direct-to-consumer, and all other appropriate retail and wholesale markets for human or animal consumption.

As used herein, the term “dosage form” includes oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings.

As used herein, the terms “extract,” “botanical extracts,” “organic extract,” and “organic liquid extract” and the like as used interchangeably herein mean and include extracts directly derived and/or prepared from plants and plant materials. Botanical extracts may be created by soaking the botanical, plant, or plant material in a liquid that is able to retrieve certain chemicals or beneficial parts of the plant to be used in a product.

As used herein, the term “buffering agent” means salts of a weak acid and a weak base that are widely used to stabilize the pH of solutions and are typically composed of weak acids and bases mixed in an aqueous solution, and can interact electrostatically with charged surfaces such as bio-membranes including for example, disodium phosphate, potassium dihydrogen phosphate, potassium chloride, sodium gluconate. The preferred buffering range of the buffering agent as used herein is in a range of between a pH of 6.5 and 8.

As used herein, the term “excipient” means non-active ingredients that act as enhancers, stabilizers, or fillers to help make the medication more effective including for example, sodium chloride, organic polysaccharide.

As used herein, the term “food additive” means any of various chemical substances added to foods to produce specific desirable effects such as salt, spices, and sulfites that are added to for instance, to preserve foods and make them more palatable including for example, sodium citrate.

As used herein, the term “stabilizer” means substances that increase stability and thickness by helping foods remain in an emulsion and retain physical characteristics and generally ingredients which normally do not mix, such as oil and water, need stabilizers, including for example, citric acid, disodium phosphate.

As used herein, the term “surfactant” means chemical compounds that decrease the surface tension or interfacial tension between two liquids, a liquid and a gas, or a liquid and a solid, and may function as emulsifiers, wetting agents, detergents, foaming agents, or dispersants, including for example, dimethicone, calcium stearate.

As used herein, the term “emollient” means substances that help soothe, soften, and increase moisture levels, especially in the skin including for example, isopropyl myristate, glycerin.

As used herein, the term “moisturizing agent” means substances that are added to cosmetics to prevent any loss of moisture and hydrate the skin and help in retaining the inherent moisture including for example, hydroxyethyl urea, isopropyl myristate, dimethicone, propylene glycol, allantoin.

As used herein, the term “emulsifier” means substances that are added to compositions etc., to prevent the separation of their oil and water components and also give these foods a smooth texture and increase their shelf life including for example, disodium phosphate, polysorbate 60, smooth and creamy lotion bar additive.

As used herein, the term “antifoaming agent” means a chemical additive that reduces and hinders the formation of foam and generally they are inert chemicals including for example, dimethicone.

As used herein, the term “solvent” means a substance that dissolves a solute, resulting in a solution including for example, propylene glycol, water, ethanol, glycerol.

As used herein, the term “coloring agent” means substances which color other substances that are devoid of color (transparent, white, or gray) or change the color of the substance that already has a different color and are generally any dye, pigment or substance, including for example, artificial coloring.

As used herein, the term “flavoring agent” means additive substances that give a dosage form or composition an additional taste or flavor including for example, citric acid, sodium citrate, natural fruit flavor.

As used herein, the term “antimicrobial” means substances that are useful for reducing and/or preventing microbial such as bacterial infection/superinfection including for example, diazolidinyl urea.

As used herein, the term “preserving agent” means additives reduce the risk of foodborne infections, decrease microbial spoilage, and preserve fresh attributes and nutritional quality including for example, citric acid, diazolidinyl urea, iodopropynyl butylcarbamate, sodium citrate, sucrose.

As used herein, the term “vitamin” means and includes water-soluble vitamins such as thiamin, riboflavin, niacin, vitamin B6, folic acid, vitamin B12, pantothenic acid, biotin, and vitamin C; and the fat-soluble vitamins such as vitamin A, vitamin E, vitamin D, and vitamin K, and including for example, retinyl palmitate, tocopherol, vitamin E acetate.

As used herein, the term “antioxidant” means substances that help to protect cellular damages from oxidative stress and also lower the risk of chronic diseases including for example, tocopherol, citric acid.

As used herein, the term “keratolytic agent” means compounds that break down the outer layers of the skin and can decrease the thickness of psoriatic plaques including for example, urea.

As used herein, the term “chelating agent” means a chemical compound that reacts with metal ions to form stable, water-soluble metal complexes and it rearranges the metal's chemical composition and improves the metal's general stability and likelihood to bond with other substances including for example, sodium gluconate, citric acid.

As used herein, the term “anti-aging agent” and/or “anti-inflammatory agent” means and includes substances that help combat the elements responsible for the visible signs of aging, and that that reduce inflammation (redness, swelling, and pain) in the body including for example, retinyl palmitate, rosemary oleoresin or rosemary oil extract, glycerin, tocopherol, citric acid.

As used herein, the term “antiseptic agent” means substances which either kill or prevent the growth of microorganisms including for example, diazolidinyl urea.

As used herein, the term “plant-derived oil” means including for example, apricot kernel (Prunus Armeniaca) oil, Macadamia ternifolia seed oil, almond or Prunus Amygdalus syn. Prunus Dulcis oil, cetearyl alcohol which is a natural fatty alcohol usually derived from palm, soy, vegetable or coconut oil, Rosmarinus officinalis (rosemary) oil, Anthemis nobilis flower oil, Chamomilla recutita (Matricaria) flower oil, raspberry oil, castor oil, rice oil, carrot seed essential oil, rosemary oleoresin or rosemary oil extract,

As used herein, the term “non-nutritive, non-digestible, water-insoluble masticatory delivery system” means and includes for example, gum base that is used to carry sweeteners, flavors, and any other substances in chewing gum and bubble gum, and it provides all the basic textural and masticatory properties of gum.

As used herein, the term “humectant” means a substance that attracts and retains the moisture in the air nearby via absorption, drawing the water vapor into or beneath the organism's or object's surface including for example, hydroxyethyl urea, propylene glycol, corn syrup, glycerin.

As used herein, the term “sweetener” means food additives used as substitutes for sugar, and may be used in such processes as food preservation, fermentation (in brewing and wine making), baking (where they contribute to texture, tenderization, and leavening), and food browning and caramelization including for example, sucrose, glucose-fructose, corn syrup, dextrose, fructose.

As used herein, the term “release agent” means and includes typically fine waxes, usually synthetic organic polymers of low molecular weight such as those of high density and medium density polyethylene, blended to desired physical properties including for example, calcium stearate.

As used herein, the term “thickening agent” means a substance which can increase the viscosity of a liquid without substantially changing its other properties including for example, organic polysaccharide, corn syrup, calcium stearate.

As used herein, the term “viral pathogen” means and includes coronaviruses, rhabdoviruses, influenza viruses, dengue viruses, severe acute respiratory syndrome coronaviruses (SARS-CoV), severe acute respiratory syndrome coronavirus 1 (SARS-COV-1), severe acute respiratory syndrome coronavirus 2 (SARS-COV-2), Middle East respiratory syndrome coronaviruses (MERS-COV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses (HSV), feline infectious peritonitis virus (FIPV), feline enteric coronavirus (FECV), rinderpest virus, foot-and-mouth disease virus (FMDV), cypoviruses (CPV), respiratory syncytial virus (RSV), human noroviruses (HuNoVs), murine norovirus (MNOV), feline calicivirus (FCV), and reoviruses.

As used herein, the term “expression” as used herein refers to transcription of DNA to produce RNA. The resulting RNA may be without limitation mRNA encoding a protein, antisense RNA that is complementary to an mRNA encoding a protein, or an RNA transcript comprising a combination of sense and antisense gene regions, such as for use in RNAi technology. “Encoding” as used herein further refers to production of encoded protein from mRNA.

As used herein, the term “viral disease” means and includes viral infection, and the resulting symptoms and pathogenesis and manifestation of the various stages and symptoms of a viral disease caused by the exposure to a viral pathogen.

As used herein, the terms “prophylactic treatment,” “prevent.” “preventing.” “prevention,” and the like refer to actions taken to decrease the chance of getting a disease or condition, particularly caused by viral pathogens as disclosed herein. It will be appreciated that, although not precluded, preventing a disease, or disorder or condition does not require that the disorder, condition or symptoms associated therewith are completely avoided and a milder form is encompassed within this definition. Thus, the prevention and the like could be complete or partial.

In any of the ranges described herein, the endpoints of the range are included in the range. However, the description also contemplates the same ranges in which the lower and/or the higher endpoint is excluded. Additional features and variations of the invention will be apparent to those skilled in tire art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

Unless defined otherwise, all technical and scientific terms used herein have the same meaning as commonly understood by one of ordinary skill in the art to which this disclosure is related. Units, prefixes, and symbols are denoted in their Systems International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. The entire document is intended to be viewed as a unified disclosure, and it should be understood that all combinations of features described herein are contemplated. All references cited herein are hereby incorporated by reference in their entireties.

Embodiments will now be described in details with reference to the accompanying drawings. To avoid unnecessarily obscuring in the present disclosure, well-known features may not be described, or substantially the same elements may not be redundantly described, for example. This is for ease of understanding. The drawings and the following description are provided to enable those skilled in the art to fully understand the present disclosure and are in no way intended to limit the scope of the present disclosure as set forth in the appended claims.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any extracts, ingredients, compositions, or methods provided herein can be combined with one or more of any of the other compositions and methods provided herein.

As discussed hereinabove, there remains a problem and need for alternate prevention and treatment strategies and mechanisms for viral infections and viral disease caused by viral pathogens such as coronaviruses, rhabdoviruses, influenza viruses, dengue viruses, severe acute respiratory syndrome coronaviruses (SARS-COV), severe acute respiratory syndrome coronavirus 1 (SARS-COV-1), severe acute respiratory syndrome coronavirus 2 (SARS-COV-2), Middle East respiratory syndrome coronaviruses (MERS-COV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses (HSV), feline infectious peritonitis virus (FIPV), feline enteric coronavirus (FECV), rinderpest virus, foot-and-mouth disease virus (FMDV), cypoviruses (CPV), respiratory syncytial virus (RSV), human noroviruses (HuNoVs), murine norovirus (MNoV), feline calicivirus (FCV), and reoviruses, etc. The existing antivirals, vaccines, and other pharmacological methodologies are insufficient and even fail to address the need of public health and management of communicable viral diseases and prevention of spread of communicable viral diseases associated with the sudden emergence of novel viral strains, mutation-driven resistance of viruses against such developed antivirals and vaccines. Thus, there is a need for alternate means and methods that can inhibit or prohibit and target viral infections and diseases at the commencement including curbing viral replication and/or viral entry into the individual hosts to stop and prevent viral disease as well as increase immunity against viral pathogens.

To address the abovementioned problems and need in the area of viral diseases which may even develop into pandemics like the recent COVID-19 pandemic, the present disclosure provides an alternate solution in the form of botanical extracts derived from plants and plant materials and combinations thereof as disclosed herein that have an ability to prevent viral binding, uptake, replication, infection, and disease.

One such botanical extract is the one derived from the brown seaweed, Laminaria japonica which is popular as “kombu” in Japanese cuisine and its main component is Fucoidan. Kwon P. S., et al., which is included here by reference had initially investigated the inhibitory pharmacodynamics of heparin and heparin like polysaccharides on belonging to a class of compounds generically know as glycosaminoglycans (GAGs) and found that sulfated polysaccharides effectively inhibit SARS-COV-2 in vitro. In this study, prior to exploring the anti-viral properties, cytotoxicity was under consideration. All compounds investigated in this referred study exhibited no cytotoxic effects at concentrations well above those that confer binding inhibition between the S-protein and the angiotensin converting enzyme 2 (ACE2) receptor binding domain (RBD). Cytotoxicity assays were carried out in Vero cells and the common toxicity assay employing water soluble tetrazolium salt-1. Further, it was interestingly observed that these GAG compounds bound more tightly to SARS-COV-2 spike proteins (S-protein) than either SARS-COV or the MERS-COV S-proteins with a KD 4-fold less and 2-fold less, respectively. The Fucoidan compound with the lowest KD, corresponding to the highest binding affinity was a high molecular weight Fucoidan derivative. At an EC50, which is a pharmacological parameter which quantifies the half maximal concentration that inhibits the binding of a ligand to a substrate.

Considering the above study, the present disclosure provides that it is the half maximal concentration in which the ACE-2 receptor is blocked by Fucoidan. Moreover, the present disclosure provides that the aforesaid bioactive compound acts as a competitive inhibitor of SARS-COV-2 and the angiotensin converting enzyme-2 (ACE-2) receptor expressed in oral, nasal, respiratory and gastrointestinal tract epithelial cells in addition to other tissues. The present invention provides that by inhibiting viral-cell binding; viral uptake, replication, and shedding is quantitatively reduced as was referred to in the abovementioned study.

Moreover, comparing to the FDA EUA medication Remdesivir, widely used in hospitals to combat SARS-COV-2 infections, the Fucoidan compound of the present invention had EC50 roughly 10-fold less, 770 nM for Remdesivir vs. ˜83 nM for the Fucoidan compound. Heparin, the principal molecule investigated has a KD exponentially less than the Fucoidan compound, 2.1 μM vs. 83 nM. The proposed higher affinity associated with the Fucoidan compound relative to heparin has been attributed to the branched molecular structure and associated higher molecular weight compared to the linear structure of heparin. It is this difference that provides additional pharmacophores available for bonding between the SARS-COV-2 spike protein and the Fucoidan molecule. Importantly, Fucoidan containing preparations can be delivered orally or nasally providing an easy route of administration. Kwon et al. had suggested further investigations needs to be carried out in epithelial cells.

In the present disclosure, starting from the aforementioned Fucoidan, substantiating and potentiating its antiviral activity in a combination with other extracts was explored so as to develop on this alternate means of preventing viral binding, infection, replication, and disease. The present invention developed and assessed a combination of botanical extracts comprising Fucoidan to empirically reach a combination of extracts derived from plant materials that is efficient in preventing the virus from replicating, binding, infecting, and causing disease and its use in the form of various end products. The present invention empirically discloses that one such effective partner to formulate an effective combination of botanical extracts with Fucoidan derived from Laminaria japonica was the extract derived from Andrographis paniculate.

Andrographis paniculata and its extract are also compounds that have been known to exhibit anti-SARS-COV-2 properties. Sa-ngiamsuntorn K. et al., investigated the anti-viral properties of Andrographis paniculata against SARS-COV-2. It is proposed that in addition to inhibition of S-protein ACE-2 binding. A. paniculata inhibits downstream regulation of RNA polymerase and 3-CL protease among others. The said mechanism carries a potential for an unrecognized additive if not synergistic effect when combined with Fucoidan. Utilizing Calu-3 lung epithelia cell lines A. paniculata extract exhibits an IC50 of 36 ng/ml with the active compound andrographolide having an IC50 of 34 nM; as compared with Remdesivir that has an IC50, in this study, of 86 nM in Calu-3 cells. The therapeutic index which examines the window between efficacy and toxicity is wide for both the extract and active compound. The CC50 for A. paniculata is greater than 100 μg/ml and for andrographolide the CC50 is 58.03 μg/ml or approaching an exponential margin of safety.

In one embodiment of the present invention, it discloses a medicinal composition for the prevention of viral binding, uptake, replication, infection, and disease caused by viral pathogens, comprising: an organic liquid extract derived from Laminaria japonica; an organic liquid extract derived from Andrographis paniculate; at least one of purified distilled water, ethanol, and glycerol; optionally at least one buffering agent; optionally at least one excipient; optionally at least one food additive; optionally at least one stabilizer; optionally at least one surfactant; optionally at least one emollient; optionally at least one moisturizing agent; optionally at least one emulsifier; optionally at least one surfactant; optionally at least one antifoaming agent; optionally at least one solvent; optionally at least one coloring agent; optionally at least one flavoring agent; optionally at least one antimicrobial; optionally at least one preserving agent; optionally at least one vitamin; optionally at least one antioxidant; optionally at least one anti-inflammatory agent; optionally at least one keratolytic agent; optionally at least one chelating agent; optionally at least one anti-aging agent; optionally at least one antiseptic agent; optionally at least one plant-derived oil; optionally at least one non-nutritive, non-digestible, water-insoluble masticatory delivery system; optionally at least one humectant; optionally at least one sweetener; optionally at least one release agent; and optionally at least one thickening agent, wherein the medicinal composition is formulated into a dosage form for the prevention of viral binding, uptake, replication, infection, and disease.

In another embodiment of the medical composition as disclosed herein, wherein the the organic liquid extract derived from Laminaria japonica is selected from a group consisting of 0.025%, 0.05%, 0.1%, 0.25%, 0.5%, 1.0%, 1.5%, 2.0%, 3.0%, 4.0%, 5.0%, 6.0%, 7.0%, 8.0%, 9.0%, or 10% of the organic liquid extract derived from Laminaria japonica in terms of mass by volume (M/V) percentage of the solution and having a biological activity of between 90% and 100% viral uptake inhibition per milliliter. Given that such agents are a mixture of polymeric sizes, the percentage inhibition of viral binding and/or uptake to epithelial cells has been disclosed herein.

In another embodiment of the medical composition as disclosed herein, wherein the organic liquid extract derived from Laminaria japonica comprises fucoidan.

In another embodiment of the medical composition as disclosed herein, wherein the organic liquid extract derived from Andrographis paniculate is selected from a group consisting of 0.025%, 0.05%, 0.1%, 0.25%, 0.5%, 1.0%, 1.5%, 2.0%, 3.0%, 4.0%, 5.0%, 6.0%, 7.0%, 8.0%, 9.0%, or 10% of the organic liquid extract derived from Andrographis paniculate in terms of mass by volume (M/V) percentage of the solution and having a biological activity of between 90% and 100% viral uptake inhibition per milliliter. Given that such agents are a mixture of polymeric sizes, the percentage inhibition of viral binding and/or uptake to epithelial cells has been disclosed herein.

In another embodiment of the medical composition as disclosed herein, wherein the organic liquid extract derived from Andrographis paniculate comprises andrographolide.

Notably, fucoidan or extract derived from Laminaria japonica and andrographolide or extract derived from Andrographis paniculate can be used up to their solubility limit as is known in the prior art.

In another embodiment of the medical composition as disclosed herein, wherein the ratio of the organic liquid extract derived from Laminaria japonica to the organic liquid extract derived from Andrographis paniculate in the medicinal composition is in a range of between 1:1000 to 1000:1 and includes a ratio of 1:1.

In another embodiment of the medical composition as disclosed herein, wherein the viral pathogens comprise coronaviruses, rhabdoviruses, influenza viruses, dengue viruses, severe acute respiratory syndrome coronaviruses (SARS-COV), severe acute respiratory syndrome coronavirus 1 (SARS-COV-1), severe acute respiratory syndrome coronavirus 2 (SARS-COV-2), Middle East respiratory syndrome coronaviruses (MERS-COV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses (HSV), feline infectious peritonitis virus (FIPV), feline enteric coronavirus (FECV), rinderpest virus, foot-and-mouth disease virus (FMDV), cypoviruses (CPV), respiratory syncytial virus (RSV), human noroviruses (HuNoVs), murine norovirus (MNoV), feline calicivirus (FCV), and reoviruses.

In another embodiment of the medical composition as disclosed herein, wherein the dosage form is selected from a group consisting of oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings.

In one embodiment of the present invention, it discloses a method for preparing the medicinal composition for the prevention of viral binding, uptake, replication, infection, and disease caused by viral pathogens of as disclosed in the present disclosure, the method comprising the steps of: (i) preparing an organic liquid extract derived from Laminaria japonica and an organic liquid extract derived from Andrographis paniculate from the respective plant comprising the following steps: obtaining a fresh plant material from a growing plant, wherein the plant is selected from Laminaria japonica and Andrographis paniculate; washing the fresh plant material with purified distilled water, air-drying it to obtain an air-dried plant material, crushing the air-dried plant material into particles of 20-mesh size to obtain crushed particles; extracting an extract from crushed particles of step (b) to obtain an organic liquid extract derived from Laminaria japonica or an organic liquid extract derived from Andrographis paniculate; (ii) measuring and combining the organic liquid extract derived from Laminaria japonica and the organic liquid extract derived from Andrographis paniculate as obtained in step (i) together and with one or more of the following ingredients to obtain a first mixture: at least one of purified distilled water, ethanol, and glycerol; optionally at least one buffering agent; optionally at least one excipient; optionally at least one food additive; optionally at least one stabilizer; optionally at least one surfactant; optionally at least one emollient; optionally at least one moisturizing agent; optionally at least one emulsifier; optionally at least one surfactant; optionally at least one antifoaming agent; optionally at least one solvent; optionally at least one coloring agent; optionally at least one flavoring agent; optionally at least one antimicrobial; optionally at least one preserving agent; optionally at least one vitamin; optionally at least one antioxidant; optionally at least one anti-inflammatory agent; optionally at least one keratolytic agent; optionally at least one chelating agent; optionally at least one anti-aging agent; optionally at least one antiseptic agent; optionally at least one plant-derived oil; optionally at least one gum base; optionally at least one humectant; optionally at least one sweetener; optionally at least one release agent; and optionally at least one thickening agent; (iii) heating the first mixture obtained in step (ii) at temperature in the range of between 140° C. and 40° C. and mixing it while heating to obtain a second mixture; (iv) cooling the second mixture obtained in step (iii) to a temperature below room temperature to obtain a third mixture; (v) processing the third mixture obtained in step (iii) into a dosage form of the medicinal composition for the prevention of viral binding, uptake, replication, infection, and disease caused by viral pathogens of as disclosed in the present disclosure, wherein the dosage form is selected from a group consisting of oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings. In another embodiment herein, the temperature below room temperature in a range of between 25° C. and 18° C. In another embodiment herein, the extraction process involves extracting an extract from the crushed particles in an acid for 2 hours; centrifuging the extract at 11,000×g, neutralizing and removing the supernatant, resuspending and centrifuging the centrifugate again to obtain an organic extract derived from Laminaria japonica as a precipitate or an organic extract derived from Andrographis paniculate as a precipitate; resuspending and diluting the precipitate with sterile, purified distilled water and passing it through a 0.20-μm filter to obtain an organic liquid extract derived from Laminaria japonica or an organic liquid extract derived from Andrographis paniculate. Alternatively, in another embodiment herein, the extraction process involves various conditions of temperature (in a range of temperature of between 80° C. and 200° C.), pressure (in a range of pressure of between 5 bar and 100 bar), and solvents (selected from a group comprising water, 0.1% sodium hydroxide, 0.1% formic acid, 70% ethanol, 50% ethanol, and 25% ethanol). Also, in another embodiment herein, an alternate for the extraction process involves combining the crushed particles with a liquid comprising at least one of water, ethanol, and glycerol; allowing the liquid to extract an organic extract at a temperature between about room temperature and simmering for about one (1) day to about sixty (60) days, or by boiling for less than about one day to form the liquid extract; and separating the liquid extract from the plant material.

In another embodiment of the method for preparing the medicinal composition as disclosed herein, wherein the heating in step (iii) comprises optionally heating in two steps, first at a higher temperature in a range of temperature between 140° C. and 85° C. and second at a lower temperature for cooling in a range of temperature between 55° C. and 40° C., wherein the two steps of heating are performed while mixing, and wherein the mixing is carried out with centrifugation or while rotating the mixture in step (iii).

In another embodiment of the method for preparing the medicinal composition as disclosed herein, wherein the mixing in step (iii) comprises optionally combining the hydrophilic ingredients separately to obtain an aqueous mixture, and combining the hydrophobic ingredients separately to obtain an oil-based mixture, and then combining said aqueous mixture and oil-based mixture together to obtain the second mixture.

In one embodiment of the present invention, it discloses a method for prophylactic treatment and prevention of viral binding, uptake, replication, infection, and disease caused by viral pathogens in an individual, the method comprising the steps of: (a) recruiting an individual; (b) testing the health and well-being of the individual to assess whether said individual is free of or has been previously exposed to or carrying a viral pathogen and/or resulting viral infection and viral disease; (c) categorizing the individual into pre-exposed or non-exposed group based on the result of testing in step (b); (d) administering or exposing the individual from step (c) to a medicinal composition as disclosed in the present disclosure in a dosage form, said medicinal composition comprising an organic liquid extract derived from Laminaria japonica and an organic liquid extract derived from Andrographis paniculate; (e) testing the health and well-being of the individual as well as performing in vitro exposure studies in epithelial cells collected from said individual to test the effectivity of administering or exposing the individual to the medicinal composition in step (d), wherein the dosage form is selected from a group consisting of oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings, wherein the dosage form including fabric for clothing and face coverings, filters for clothing and face coverings, and coatings for clothing and face coverings is directed to prevent the inhalation and contact with airborne and surface viral pathogens, wherein the dosage form including diffusers is directed against surface and airborne viral pathogens, wherein the medicinal composition in the dosage form prevents the binding and uptake of viral pathogens by oral and nasal epithelial cells, and wherein additive, potentiating, and synergistic effects of various ingredients in the medicinal composition as combined increase the efficacy of a single ingredient as compared with the efficacy of the single ingredient alone, wherein the single ingredient is selected from a group consisting of the organic liquid extract derived from Laminaria japonica and the organic liquid extract derived from Andrographis paniculate.

In another embodiment of the method for prophylactic treatment and prevention as disclosed herein, wherein the organic liquid extract derived from Laminaria japonica is selected from a group consisting of 0.025%, 0.05%, 0.1%, 0.25%, 0.5%, 1.0%, 1.5%, 2.0%, 3.0%, 4.0%, 5.0%, 6.0%, 7.0%, 8.0%, 9.0%, or 10% of the organic liquid extract derived from Laminaria japonica in terms of mass by volume (M/V) percentage of the solution and having a biological activity of between 90% and 100% viral uptake inhibition per milliliter. Given that such agents are a mixture of polymeric sizes, the percentage inhibition of viral binding and/or uptake to epithelial cells has been disclosed herein.

In another embodiment of the method for prophylactic treatment and prevention as disclosed herein, wherein the organic liquid extract derived from Laminaria japonica comprises fucoidan.

In another embodiment of the method for prophylactic treatment and prevention as disclosed herein, wherein the organic liquid extract derived from Andrographis paniculate is selected from a group consisting of 0.025%, 0.05%, 0.1%, 0.25%, 0.5%, 1.0%, 1.5%, 2.0%, 3.0%, 4.0%, 5.0%, 6.0%, 7.0%, 8.0%, 9.0%, or 10% of the organic liquid extract derived from Andrographis paniculate in terms of mass by volume (M/V) percentage of the solution and having a biological activity of between 90% and 100% viral uptake inhibition per milliliter. Given that such agents are a mixture of polymeric sizes, the percentage inhibition of viral binding and/or uptake to epithelial cells has been disclosed herein.

In another embodiment of the method for prophylactic treatment and prevention as disclosed herein, wherein the organic liquid extract derived from Andrographis paniculate comprises andrographolide.

Notably, fucoidan or extract derived from Laminaria japonica and andrographolide or extract derived from Andrographis paniculate can be used up to their solubility limit as is known in the prior art.

In another embodiment of the method for prophylactic treatment and prevention as disclosed herein, wherein the ratio of the organic liquid extract derived from Laminaria japonica to the organic liquid extract derived from Andrographis paniculate in the medicinal composition is in a range of between 1:1000 to 1000:1 and includes a ratio of 1:1.

In another embodiment of the method for prophylactic treatment and prevention as disclosed herein, wherein the viral pathogens comprise coronaviruses, rhabdoviruses, influenza viruses, dengue viruses, severe acute respiratory syndrome coronaviruses (SARS-COV), severe acute respiratory syndrome coronavirus 1 (SARS-COV-1), severe acute respiratory syndrome coronavirus 2 (SARS-COV-2), Middle East respiratory syndrome coronaviruses (MERS-COV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses (HSV), feline infectious peritonitis virus (FIPV), feline enteric coronavirus (FECV), rinderpest virus, foot-and-mouth disease virus (FMDV), cypoviruses (CPV), respiratory syncytial virus (RSV), human noroviruses (HuNoVs), murine norovirus (MNoV), feline calicivirus (FCV), and reoviruses.

In another embodiment of the method for prophylactic treatment and prevention as disclosed herein, wherein the individual is additionally administered or exposed to one or more antivirals, wherein antivirals comprise Remdesivir, Nirmatrelvir with Ritonavir (Paxlovid), and Molnupiravir.

In another embodiment of the method for prophylactic treatment and prevention as disclosed herein, wherein the individual comprises human beings, domesticated animals, farm animals, companion animals, livestock, zoo animals, agricultural beasts, and wild animals.

Thus, the object of the present invention is to provide and alternate means in the form of a combination of materials which are botanical extracts that act with strong additivity and synergy in preventing a viral pathogen from replicating, binding, infecting, and ultimately causing a viral disease and the same is used in various forms and formulations as dosage forms as disclosed herein to produce various end products for use in human and animal consumption.

The invention will be further explained by the following Examples, which are intended to purely exemplary of the invention, and should not be considered as limiting the invention in any way.

### EXAMPLES

### Example 1

### Determination of Spike Protein Binding to Human Oral Epithelial Cells:

### Human Oral Epithelial Cell Collection and Preparation:

In this example, first, human volunteer subjects fast for at least 30 minutes prior to cell collection. After rinsing the mouth with water, the subjects gargled with saline for 60 seconds. The saline was then ejected into a funnel and a 10 ml Falcon tube. The cells were then centrifuged at 600×g for 10 min. The supernatant was then removed from above the cell pellet and cells in said cell pellet were resuspended in 10 ml PBS and centrifuged again at 3000×g for 10 min. The supernatant was then removed and the cells were resuspended in 2 ml PBS and the samples were split to 100 μl aliquots in 1.5 ml microcentrifuge tubes to obtain various cell preparations of human oral epithelial cells for further procedures and experimentations.

### Coronavirus Spike Protein Uptake Procedure:

Next, in this example, coronavirus spike protein was added to the cell preparation as prepared hereinabove for a final concentration of 1.0 ng-1.0 μg/ml. Cells were incubated for 1-12 hours at 37° C. with moderate agitation (90 RPM on an orbital shaker). Following incubation with the spike protein, tubes were centrifuged at 4000×g and washed with at least 1.5 ml PBS for a total of 3 washes and then fixed. Cells were fixed and permeabilized using 1 ml per tube absolute methanol, or ethanol, acetone can also be used. The tubes were then placed in a −20° C. freezer for 10 min. Following fixation cells were washed once with PBS. Nonspecific binding sites were then blocked using a solution of 3% bovine serum albumin at room temperature for at least one hour, with moderate agitation. Cells were then labelled with Abclonal (Cat NO. A20135) anti-SP 1:2000 for one hour with mild agitation at room temperature and then washed two times with PBS, as above. Then, HRP-labelled secondary antibody was added for 30 min and the cells were then washed three times as above. The cells were then plated onto a flat bottom 96 well plate at 50-100 μl per sample, with the cell density adjusted so that single cells can be easily counted. 50 μl membrane peroxidase substrate (Seracare) was then added. Spike protein binding and uptake was quantified on a photograph from the microscope taken at 100× by counting positive and negative stained cells in 3 wells for each sample. Statistical analysis (t-test, ANOVA or Chi Square) was then applied to the cell counts. Standard errors were calculated for graphs of the data.

### Example 2

In Vitro Potency of Fucoidan Derived from Laminaria japonica:

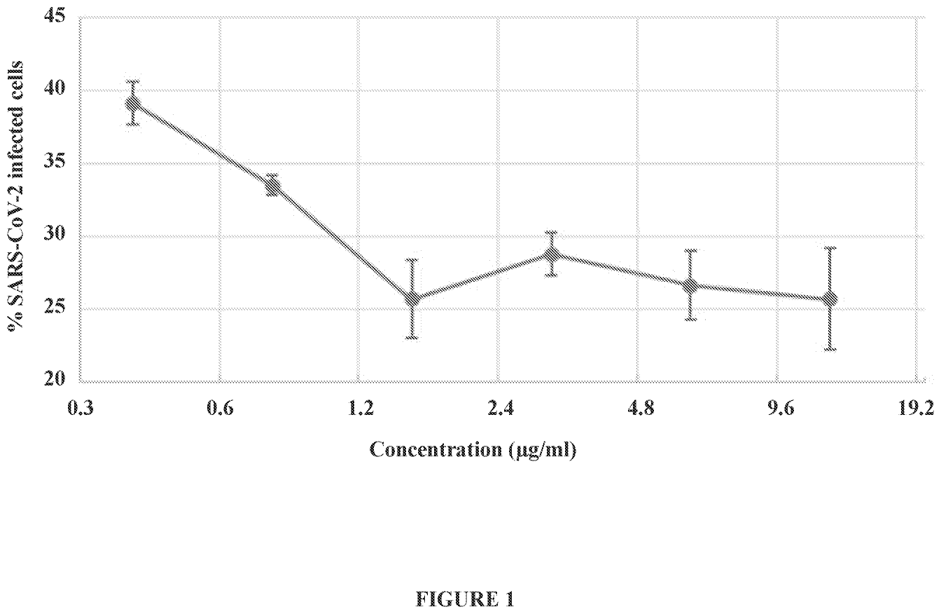

In this example, the cell preparation was prepared as disclosed above in Example 1 for primary oral epithelial cells after they were collected from healthy volunteer subjects and treated with 1 μg/ml COVID-19 causing viral pathogen, SARS-COV-2's spike protein and 0-50 μg fucoidan derived from Laminaria japonica for 1 hour at 37° C. The cell preparation was then washed three times and fixed with methanol. Immunocytochemistry was performed as described hereinabove and the cells were counted and the percentage of COVID-19-positive cells was calculated. From this example, as graphically shown in FIG. 1 disclosed herein, Fucoidan inhibited spike protein uptake with an IC50 of approximately 1 μg/ml.

### Example 3

In Vivo Dose Response Fucoidan Derived from Laminaria japonica:

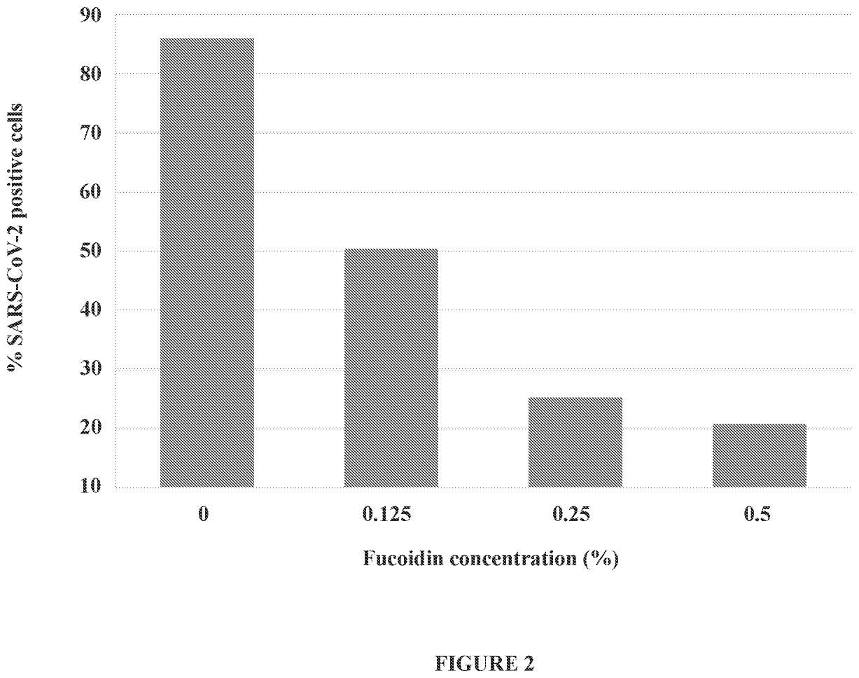

In this example, primary human oral epithelial cells were collected from a healthy volunteer subject after three 200 μl sprays of saline or saline containing 0.125-0.5% fucoidan derived from Laminaria japonica. The dose was doubled every 30 minutes and cells were again collected. Cells were incubated with COVID-19 causing viral pathogen, SARS-COV-2's spike protein at 1 μg/ml for 1 hour at 37° C. and then washed three times and fixed and permeabilized using methanol. Immunocytochemistry was performed as described hereinabove and the cells were counted and the percentage of COVID-19-positive cells was calculated. This example as graphically demonstrated in FIG. 2 disclosed herein, establishes in vivo that Fucoidan inhibited spike protein uptake with an IC50 of approximately 0.125%.

### Example 4

Transmission Study and COVID-19 Infection in the Absence (Pre) and Presence (Post) of Fucoidan Derived from Laminaria japonica:

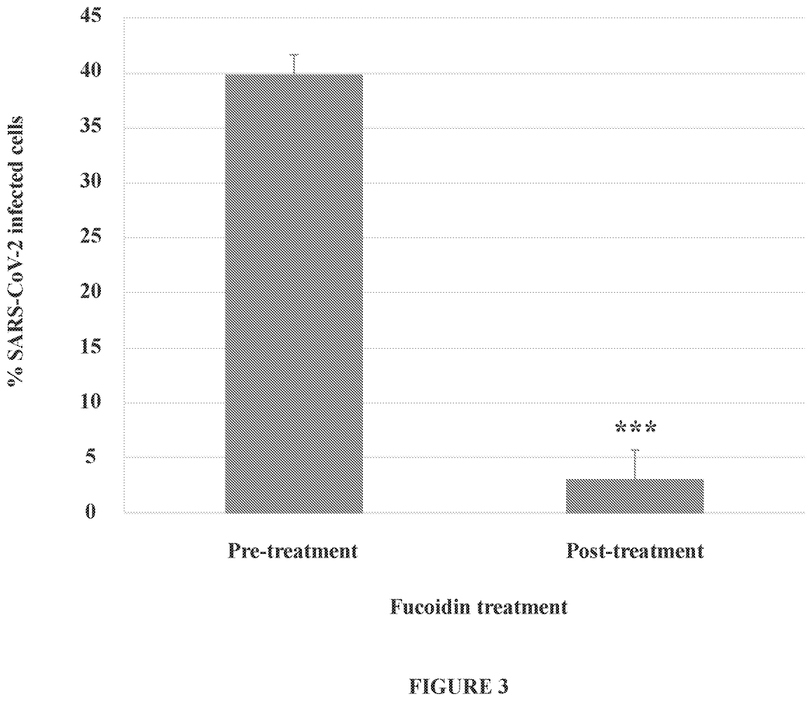

In this example, three healthy volunteer subjects who were negative for COVID-19 were asked to do an oral rinse with COVID-19 causing viral pathogen, SARS-COV-2's spike protein (where a 10 ml rinse solution had a 1 μg/ml solution of spike protein in saline). The subjects were then instructed to cough toward a petri dish containing primary human oral epithelial cells (prepared as provided in Example 1) from a distance of 1 meter. The volunteers then used a fucoidan (derived from Laminaria japonica) safe sweet lozenge and rinsed again with the spike protein. They again coughed toward a petri dish containing primary human oral epithelial cells. The cells were then harvested and immunocytochemistry was performed on the pre-fucoidan treatment/exposure samples (pre) and post-fucoidan treatment/exposure samples as graphically shown in FIG. 3 by a process as provided hereinabove in Example 1. As inferred from this example and results in FIG. 3, fucoidan treatment/exposure (post bar in FIG. 3) showed a sharp and significant decline in the percentage of COVID-19-infected cells.

### Example 5

In Vitro Dose Response Andrographolide Derived from Andrographis paniculate:

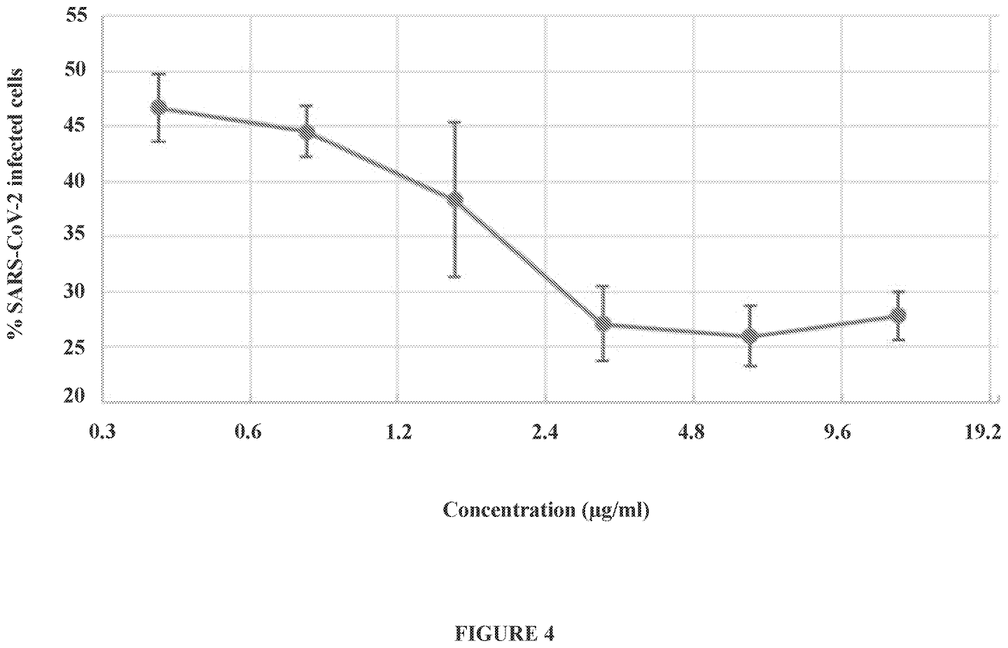

In this example, primary oral epithelial cells were collected from healthy volunteer subjects in a similar procedure as disclosed above in Example 1 and treated with 1 μg/ml COVID-19 causing viral pathogen, SARS-COV-2's spike protein and 0-2 μg andrographis extract derived from Andrographis paniculate for 1 hour at 37° C. The cell preparation was then washed three times and fixed with methanol. Immunocytochemistry was performed as described hereinabove in Example 1 and the cells were counted and the percentage of COVID-19-positive cells was calculated. This example and the results as graphically disclosed in FIG. 4 provide that andrographis inhibited spike protein uptake with an IC50 of approximately 0.125 μg/ml.

### Example 6

Strong Additive and Synergistic Effect when Combining Fucoidan Derived from Laminaria japonica and Andrographolide or Andrographis Extract Derived from Andrographis paniculate:

In this example, primary oral epithelial cells were collected from healthy volunteer subjects as shown in Example 1 and treated with 1 μg/ml COVID-19 causing viral pathogen, SARS-COV-2's spike protein, along with 1 μg/ml fucoida derived from Laminaria japonica, and andrographis extract derived from Andrographis paniculate at 0-0.8 μg/ml for 1 hour at 37° C. The cell preparation in this example was then prepared as provided in Example 1 and then washed three times and fixed with methanol. Immunocytochemistry was performed as described in Example 1 hereinabove and the cells were counted and the percentage of COVID-19-positive cells was calculated. This example and the results and data shown in the Table 1 hereinbelow shows that a combination of fucoidan and andrographis showed strong additivity and possibly synergetic effect over and above individual extract's effect in the inhibition of COVID-19 causing viral pathogen, SARS-COV-2's spike protein uptake.

TABLE 1
Interaction of Fucoidan derived from Laminaria japonica
and Andrographolide or andrographis extract derived
from Andrographis paniculate:

| Andrographis | Fucoidan | | |
|---|---|---|---|
| (μg/ml) | (μg/ml) | Mean | SEM |
| 0 | 1 | 33 | — |
| 0.2 | 1 | 31.60333 | 2.32812* |
| 0.4 | 1 | 30.65333 | 1.33462* |
| 0.8 | 1 | 26.49667 | 2.0372** |

*P < 0.001 vs vehicle.
**P < 0.001 vs vehicle,
P < 0.05 vs 1 ug/ml fucoidan alone

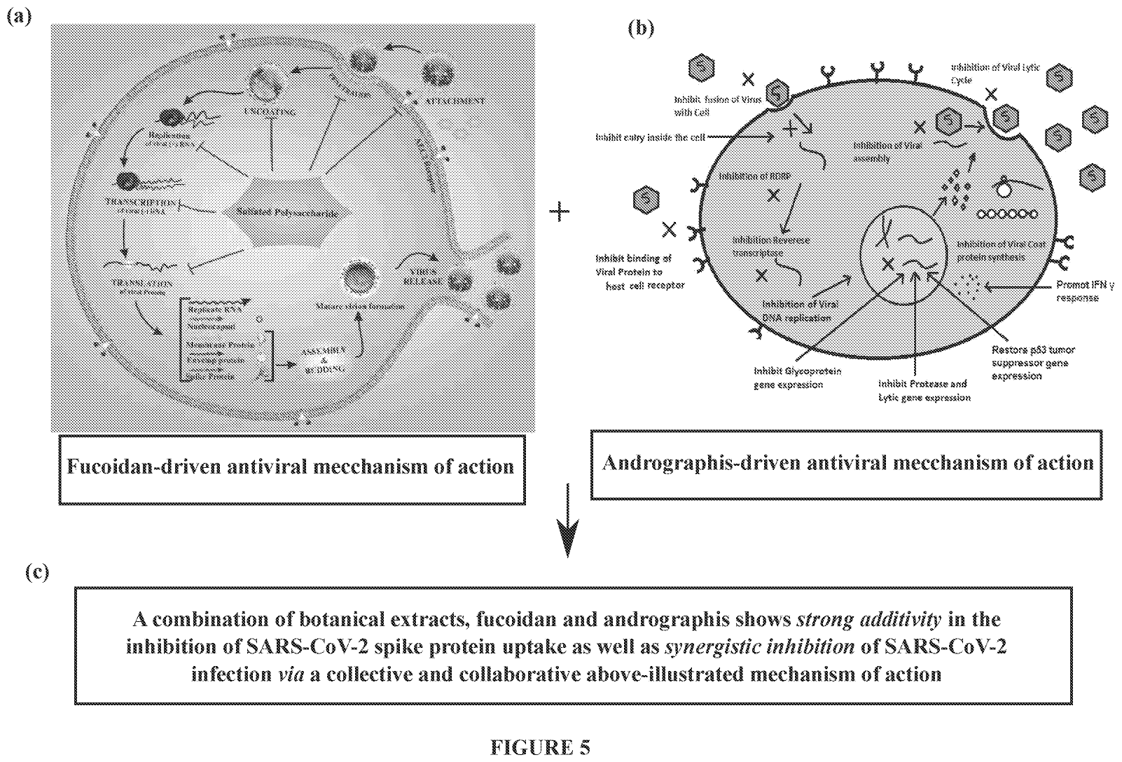

Further, FIG. 5 provides a schematic diagram of (a) sulfated polysaccharides (SPs) such as fucoidan modulating antiviral mechanism of via inhibiting virus attachment, penetration, interiorization, uncoating and transcription and translation process when combined with (b) mode of action of andrographolides on viral replication in animal cell showing a strong additive and/or synergistic effect on the inhibition of COVID-19 by targeting SARS-COV-2's spike protein.

### Example 7

Formulation of Medicinally Acceptable Preparations and Medicinal Compositions in Dosage Forms Formed of Organic Extracts Comprising Fucoidan Derived from Laminaria japonica and Andrographolide or Andrographis Extract Derived from Andrographis paniculate:

In this example, the below examples provide a selected variation and detailed methods for forming various dosage forms and embodiments as non-limiting examples of the present disclosure:

### Oral Spray:

Ingredients: Purified Water, Fucoidan derived from Luminaria, Andrographis extract, Citric Acid [Flavor optional-reduce purified water by 0.5%; add flavor 0.5%]

Measure and mix dry ingredients completely. Measure and add ½ distilled water at temperature 85-95 degrees Celsius and mix with vented cover at 5-7 rpm for 15 minutes. Add the remaining water to the mixture and reduce the heat to 50 degrees Celsius. Continue mixing for 20 minutes. Let cool to room temperature. Fill oral spray bottles, cover, cap and seal.

### Nasal Mist:

Ingredients: Distilled Water, Fucoidan derived from Luminaria, Andrographis extract, Sodium Chloride, Disodium Phosphate, Potassium dihydrogen phosphate, Potassium chloride. Measure and mix dry ingredients completely. Measure and add ½ distilled water at temperature 85-95 degrees Celsius and mix with vented cover at 5-7 rpm for 15 minutes. Add the remaining water to the mixture and reduce the heat to 50 degrees Celsius. Continue mixing for 20 minutes. Let cool to room temperature. Fill nasal spray bottles, cover, cap and seal.

### Barrier Hand Cream:

Ingredients: Purified Water, Apricot Kernel (Prunus Armeniaca) Oil, Macadamia Ternifolia Seed oil, Prunus Amygdalus Dulcis, Cetearyl Alcohol (and) Polysorbate 60, Organic Polysaccharide, hydroxyethyl urea, isopropyl myristate, Dimethicone, Propylene Glycol, Diazolidinyl Urea, Iodopropynyl Butylcarbamate, Retinyl Palmitate, tocopherol, Rosmarinus Officinalis, Allantoin, Sodium Gluconate, Citric Acid, Sodium Citrate, Anthemis nobilis flower oil, Chamomilla recutita (Matricaria) flower oil.

Measure and combine Purified Water, Fucoidan derived from Luminaria, Andrographis extract, and Allantoin mix heat to 50 degrees Celsius. Set aside.

Mix and measure Apricot Kernel (Prunus Armeniaca) Oil, Macadamia Ternifolia Seed oil, Prunus Amygdalus Dulcis and heat until melted. Separately measure and mix hydroxyethyl urea, Isopropyl myristate, Dimethicone, Propylene Glycol, Diazolidinyl Urea, Iodopropynyl Butylcarbamate, Retinyl Palmitate, tocopherol, Rosmarinus Officinalis, Anthemis nobilis flower oil, Chamomilla recutita (Matricaria) flower oil. Mix at 3000 rpm and melt to 50 degrees Celsius, Set aside.

When water mixture and oil mixture are less than 50 degrees but greater than 40 degrees Celsius combine the mixtures to create a milky mixture. Some evaporation will take place at this mixing phase, add sufficient purified water when cooled below 40% Celsius.

Fill 50 g pump bottles, cap and seal.

### Prophylactic Lozenges:

Ingredients: Sucrose, purified water, glucose-fructose, Fucoidan derived from Luminaria, Andrographis extract, natural fruit flavor, artificial coloring.

Mix and measure sucrose, purified water, glucose-fructose, and organic extracts. Heat uncovered to 100 degrees C. Completely cover for 4 minutes. Uncover and heat until temperature of mixture reaches 139 degrees C. Remove from heat and add flavor and color. Pour heated mixture into 4 g molds. Cool, crack, trim, wrap and package.

### Lip Ointment:

Measure water, Fucoidan derived from Luminaria, mix and bring to a boil. Combine Raspberry Oil, Castor Oil, Macadamia Oil, Apricot Oil, Rice Oil and melt to 125 degrees C. Cool both mixtures to 50 degrees C. Mix completely. Add Smooth and creamy lotion bar additive, Vitamin E acetate, Carrot Seed Essential oil, Rosemary Oleoresin, Raspberry flavor to the combined mixture. Mix well at 5000-7000 rpm. Refrigerate for 2 hours. Fill 1 oz. tubes for lip application.

### Chewing Gum:

Melt Gum Base, Citric Acid, Corn Syrup, Glycerin, Fucoidan derived from Luminaria, Andrographis extract. Mix completely add flavor and coloring. Combine melted ingredients with powdered sugar and knead until all ingredients are combined. Roll the gum into a long rope and cut into bite size pieces.

### Prophylactic Pressed Powder Tablet:

Combine Dextrose, Fucoidan derived from Luminaria, Andrographis extract, Citric Acid, Calcium Stearate, color and flavor. Add combined mixture to tablet press. Release 1-gram tablets for pressing.

### Safe Powder Beverage Mix:

Sugar, Fructose, Citric Acid, Fucoidan derived from Luminaria, Andrographis extract, colored powder, flavor powder. Combine all ingredients and mix thoroughly. Store in airtight container. Shake before use. Use 26 g [2 TBS] of mixture per 12 fluid oz. of water. Pour 4 oz of hot water and combine with powder. Mix well. Add 8 oz of cold water and mix completely. Pour over ice before drinking.

### Safe Ice Pops:

Combine water, Fucoidan derived from Luminaria, Andrographis extract, bring to a boil. Add color and flavor. Mix completely. Cool completely. Put in 1.5 oz plastic sleeve and hot seal. Place in freezer for 6 hours prior to use. Alternatively, put in molds and place in freezer. Remove from molds and package for later use. Keep frozen until use.

### Safe Diffuser:

Combine water, Fucoidan derived from Luminaria, Andrographis extract, and bring to a boil. Let cool completely. Add essential oil and mix. Add 25% mixture with 75% purified water to diffuser. Release ingredients through diffuser for 4-8 hours.

In addition to the examples and experiments disclosed above, other experiments have been done with other sea weeds and although they kind of worked but such combinations did not meet all the other desired attributes. For instance, with rock kelp, although it was effective against viral pathogens but it had a very high fish odor making it incompatible with making a nasal spray or room diffuser or lozenge. Others like Ashwagandha (Withania somnifera), Giloy (Tinospora cordifolia) also did not provide an optimal combination considering all the functional and aesthetic attributes considering human and animal consumption. Some did not have the same level of effectiveness and some were offensive in terms of smell or odor, while others inhibited the antiviral effect of fucoidan. For instance, Aspergillus spp. (itself has some antiviral effect) but when combined with Fucoidan, they cancelled each other out. Moreover, there were failed chewing gum because the dispersion was not there as also with working out some steps for making the hard candy lozenge that are not obvious but necessary.

Even animal model experimentation will be conducted using equine or bovine epithelial cells they both have the same composition as human oral epithelial cells as used and discussed in the experiments and examples disclosed hereinabove given that there is a strong homology between human, bovine, and equine ACE-2 receptors for effectiveness of the experimental combination similar to human cells on said animal cells.

In addition, the combination as disclosed when tested on bacterial infections provided another set of negative data as it was non-effective as a combination against bacterial infections.

Further, the experimentation and use of human primary oral epithelial cells provide a pertinent assay system for in vivo human studies with the proposed dosage forms as described hereinabove and such a leap from human cells to humans is understandable, extrapolatable, and manipulable by an ordinary person of skill in the art. Also, the end product is basically a topical product, where it works on cell surface and otherwise there is no systemic absorption, it does not need to get into the bloodstream. The medicinal composition as disclosed in the present invention is applied on the epithelial cells, which is often the main route of viral replication. So, the experiments done in vitro with primary human cells is the same as spraying in your mouth and collecting human cells and they replicate what would be going on in the human mouth.

Moreover, the advantageous combination gains its advantageous effectivity because of the method of formulating a combination involving the organic extracts being brought to high temperatures in a solution that allows the combination to be more effective.

Additionally, the combination as presently disclosed and claimed also provides the advantage of meeting and being effective against a much wider variety of viruses if illustrated as a Venn diagram to show that there are effectivities outside the range of either one of the components i.e., the two botanical extracts as disclosed and used herein, and at the intersection where they show synergistic or strongly additive effectivity (refer to FIG. 5 as disclosed herein) and the range of protection is enhanced when the two are combined.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. A medicinal composition for the prevention of viral binding, uptake, replication, infection, and disease caused by viral pathogens, comprising:
an organic liquid extract derived from Laminaria japonica;
an organic liquid extract derived from Andrographis paniculate;
at least one of purified distilled water, ethanol, and glycerol;
optionally at least one buffering agent;
optionally at least one excipient;
optionally at least one food additive;
optionally at least one stabilizer;
optionally at least one surfactant;
optionally at least one emollient;
optionally at least one moisturizing agent;
optionally at least one emulsifier;
optionally at least one surfactant;
optionally at least one antifoaming agent;
optionally at least one solvent;
optionally at least one coloring agent;
optionally at least one flavoring agent;
optionally at least one antimicrobial;
optionally at least one preserving agent;
optionally at least one vitamin;
optionally at least one antioxidant;
optionally at least one anti-inflammatory agent;
optionally at least one keratolytic agent;
optionally at least one chelating agent;
optionally at least one anti-aging agent;
optionally at least one antiseptic agent;
optionally at least one plant-derived oil;
optionally at least one gum base;
optionally at least one humectant;
optionally at least one sweetener;
optionally at least one release agent; and
optionally at least one thickening agent,
wherein the medicinal composition is formulated into a dosage form for the prevention of viral binding, uptake, replication, infection, and disease.

2. The medicinal composition of claim 1, wherein the organic liquid extract derived from Laminaria japonica is selected from a group consisting of 0.025%, 0.05%, 0.1%, 0.25%, 0.5%, 1.0%, 1.5%, 2.0%, 3.0%, 4.0%, 5.0%, 6.0%, 7.0%, 8.0%, 9.0%, or 10% of the organic liquid extract derived from Laminaria japonica in terms of mass by volume (M/V) percentage of the solution and having a biological activity of between 90% and 100% viral uptake inhibition per milliliter.

3. The medicinal composition of claim 1, wherein the organic liquid extract derived from Laminaria japonica comprises fucoidan.

4. The medicinal composition of claim 1, wherein the organic liquid extract derived from Andrographis paniculate is selected from a group consisting of 0.025%, 0.05%, 0.1%, 0.25%, 0.5%, 1.0%, 1.5%, 2.0%, 3.0%, 4.0%, 5.0%, 6.0%, 7.0%, 8.0%, 9.0%, or 10% of the organic liquid extract derived from Andrographis paniculate in terms of mass by volume (M/V) percentage of the solution and having a biological activity of between 90% and 100% viral uptake inhibition per milliliter.

5. The medicinal composition of claim 1, wherein the organic liquid extract derived from Andrographis paniculate comprises andrographolide.

6. The medicinal composition of claim 1, wherein the ratio of the organic liquid extract derived from Laminaria japonica to the organic liquid extract derived from Andrographis paniculate in the medicinal composition is in a range of between 1:1000 to 1000:1 and includes a ratio of 1:1.

7. The medicinal composition of claim 1, wherein the viral pathogens comprise coronaviruses, rhabdoviruses, influenza viruses, dengue viruses, severe acute respiratory syndrome coronaviruses (SARS-COV), severe acute respiratory syndrome coronavirus 1 (SARS-COV-1), severe acute respiratory syndrome coronavirus 2 (SARS-COV-2), Middle East respiratory syndrome coronaviruses (MERS-COV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses (HSV), feline infectious peritonitis virus (FIPV), feline enteric coronavirus (FECV), rinderpest virus, foot-and-mouth disease virus (FMDV), cypoviruses (CPV), respiratory syncytial virus (RSV), human noroviruses (HuNoVs), murine norovirus (MNoV), feline calicivirus (FCV), and reoviruses.

8. The medicinal composition of claim 1, wherein the dosage form is selected from a group consisting of oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings.

9. A method for preparing the medicinal composition for the prevention of viral binding, uptake, replication, infection, and disease caused by viral pathogens of claim 1, the method comprising the steps of:
(i) preparing an organic liquid extract derived from Laminaria japonica and an organic liquid extract derived from Andrographis paniculate from the respective plant comprising the following steps:
obtaining a fresh plant material from a growing plant, wherein the plant is selected from Laminaria japonica and Andrographis paniculate;
washing the fresh plant material with purified distilled water, air-drying it to obtain an air-dried plant material, crushing the air-dried plant material into particles of 20-mesh size to obtain crushed particles;
extracting an extract from crushed particles of step (b) to obtain an organic liquid extract derived from Laminaria japonica or an organic liquid extract derived from Andrographis paniculate;
(ii) measuring and combining the organic liquid extract derived from Laminaria japonica and the organic liquid extract derived from Andrographis paniculate as obtained in step (i) together and with one or more of the following ingredients to obtain a first mixture:
at least one of purified distilled water, ethanol, and glycerol;
optionally at least one buffering agent;
optionally at least one excipient;
optionally at least one food additive;
optionally at least one stabilizer;
optionally at least one surfactant;
optionally at least one emollient;
optionally at least one moisturizing agent;
optionally at least one emulsifier;
optionally at least one surfactant;
optionally at least one antifoaming agent;
optionally at least one solvent;
optionally at least one coloring agent;
optionally at least one flavoring agent;
optionally at least one antimicrobial;
optionally at least one preserving agent;
optionally at least one vitamin;
optionally at least one antioxidant;
optionally at least one anti-inflammatory agent;
optionally at least one keratolytic agent;
optionally at least one chelating agent;
optionally at least one anti-aging agent;
optionally at least one antiseptic agent;
optionally at least one plant-derived oil;
optionally at least one gum base;
optionally at least one humectant;
optionally at least one sweetener;
optionally at least one release agent; and
optionally at least one thickening agent;
(iii) heating the first mixture obtained in step (ii) at temperature in the range of between 140° C. and 40° C. and mixing it while heating to obtain a second mixture;
(iv) cooling the second mixture obtained in step (iii) to a temperature below room temperature to obtain a third mixture;
(v) processing the third mixture obtained in step (iii) into a dosage form of the medicinal composition for the prevention of viral binding, uptake, replication, infection, and disease caused by viral pathogens of claim 1,
wherein the dosage form is selected from a group consisting of oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings.

10. The method for preparing the medicinal composition of claim 9, wherein the mixing in step (iii) comprises optionally combining the hydrophilic ingredients separately to obtain an aqueous mixture, and combining the hydrophobic ingredients separately to obtain an oil-based mixture, and then combining said aqueous mixture and oil-based mixture together to obtain the second mixture.

11. A method for prophylactic treatment and prevention of viral binding, uptake, replication, infection, and disease caused by viral pathogens in an individual, the method comprising the steps of:
(a) recruiting an individual;
(b) testing the health and well-being of the individual to assess whether said individual is free of or has been previously exposed to or carrying a viral pathogen and/or resulting viral infection and viral disease;
(c) categorizing the individual into pre-exposed or non-exposed group based on the result of testing in step (b);
(d) administering or exposing the individual from step (c) to a medicinal composition of claim 1 in a dosage form, said medicinal composition comprising an organic liquid extract derived from Laminaria japonica and an organic liquid extract derived from Andrographis paniculate;
(e) testing the health and well-being of the individual as well as performing in vitro exposure studies in epithelial cells collected from said individual to test the effectivity of administering or exposing the individual to the medicinal composition in step (d),
wherein the dosage form is selected from a group consisting of oral sprays, oral lozenges, lollipops, oral sprays, chewing gums, pressed powder tablets, lip balms, lip ointments, hand creams, decoctions, granules, ointments, pills, powders, powder beverage mixes, ice popsicles, diffusers, eye drops, nasal mists, nasal sprays, fabric for clothing and face coverings, filters for clothing and face coverings, and coatings for clothing and face coverings,
wherein the dosage form including fabric for clothing and face coverings, filters for clothing and face coverings, and coatings for clothing and face coverings is directed to prevent the inhalation and contact with airborne and surface viral pathogens,
wherein the dosage form including diffusers is directed against surface and airborne viral pathogens,
wherein the medicinal composition in the dosage form prevents the binding and uptake of viral pathogens by oral and nasal epithelial cells, and
wherein additive, potentiating, and synergistic effects of various ingredients in the medicinal composition as combined increase the efficacy of a single ingredient as compared with the efficacy of the single ingredient alone, wherein the single ingredient is selected from a group consisting of the organic liquid extract derived from Laminaria japonica and the organic liquid extract derived from Andrographis paniculate.

12. The method for prophylactic treatment and prevention of claim 11, wherein the organic liquid extract derived from Laminaria japonica is selected from a group consisting of 0.025%, 0.05%, 0.1%, 0.25%, 0.5%, 1.0%, 1.5%, 2.0%, 3.0%, 4.0%, 5.0%, 6.0%, 7.0%, 8.0%, 9.0%, or 10% of the organic liquid extract derived from Laminaria japonica in terms of mass by volume (M/V) percentage of the solution and having a biological activity of between 90% and 100% viral uptake inhibition per milliliter.

13. The method for prophylactic treatment and prevention of claim 11, wherein the organic liquid extract derived from Laminaria japonica comprises fucoidan.

14. The method for prophylactic treatment and prevention of claim 11, wherein the organic liquid extract derived from Andrographis paniculate is selected from a group consisting of 0.025%, 0.05%, 0.1%, 0.25%, 0.5%, 1.0%, 1.5%, 2.0%, 3.0%, 4.0%, 5.0%, 6.0%, 7.0%, 8.0%, 9.0%, or 10% of the organic liquid extract derived from Andrographis paniculate in terms of mass by volume (M/V) percentage of the solution and having a biological activity of between 90% and 100% viral uptake inhibition per milliliter.

15. The method for prophylactic treatment and prevention of claim 11, wherein the organic liquid extract derived from Andrographis paniculate comprises andrographolide.

16. The method for prophylactic treatment and prevention of claim 11, wherein the ratio of the organic liquid extract derived from Laminaria japonica to the organic liquid extract derived from Andrographis paniculate in the medicinal composition is in a range of between 1:1000 to 1000:1 and includes a ratio of 1:1.

17. The method for prophylactic treatment and prevention of claim 11, wherein the viral pathogens comprise coronaviruses, rhabdoviruses, influenza viruses, dengue viruses, severe acute respiratory syndrome coronaviruses (SARS-COV), severe acute respiratory syndrome coronavirus 1 (SARS-COV-1), severe acute respiratory syndrome coronavirus 2 (SARS-COV-2), Middle East respiratory syndrome coronaviruses (MERS-COV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses (HSV), feline infectious peritonitis virus (FIPV), feline enteric coronavirus (FECV), rinderpest virus, foot-and-mouth disease virus (FMDV), cypoviruses (CPV), respiratory syncytial virus (RSV), human noroviruses (HuNoVs), murine norovirus (MNoV), feline calicivirus (FCV), and reoviruses.

18. The method for prophylactic treatment and prevention of claim 11, wherein the individual is additionally administered or exposed to one or more antivirals, wherein antivirals comprise Remdesivir, Nirmatrelvir with Ritonavir (Paxlovid), and Molnupiravir.

19. The method for prophylactic treatment and prevention of claim 11, wherein the individual comprises human beings, domesticated animals, farm animals, companion animals, livestock, zoo animals, agricultural beasts, and wild animals.
